(19) United States
(12) Patent Application Publication
(10) Pub. No.: US 2003/0236444 A1
(43) Pub. Date: Dec. 25, 2003

(54) External remedy for impotence

(76) Inventor: Edgar Henry Brown JR., Newton, MA (US)

Correspondence Address: Edgar Brown, 32 Fisher Ave., Newton, MA, 02461, US

(21) Appl. No.: 10/174,474

(22) Filed: Jun. 19, 2002

(51) Int. Cl. A61F 5/00

(52) U.S. Cl. 600/38

(57) ABSTRACT

As an aid in remedying impotence (erectile dysfunction), this invention consists of a cord and arrangements for it to be wound around a man's body and secured in place and so arranged that it passes across and presses in on the upper half of his penis where his penis meets his body thus including a very simple method of restricting the flow of blood out of the penis without interfering with the blood flow into the penis nor with ejaculation. The advantage of this invention over the previous art, which consist of various rigid contraptions, is that it is much simpler, much less conspicuous, is much more easily and quickly installed, is of greater help in a man's achieving and maintaining an erect penis and has a very low cost of manufacture.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

By impotence in a man we mean the failure of sexual stimuli to induce an erection of his penis or to sustain an erection during sexual activities including successful coitus. This condition is currently also called erectile dysfunction.

Over fifty patents have been granted for various remedies for impotence. A number of these remedies consist of apparatus to apply pressure to the base of the penis, for example, a ring encircling the penis at its base. Of these, U.S. Pat. Nos. 6,074,341, 5,336,157, 3,759,020, 3,612,046, and 3,511,230, achieve their effect by pressing against a portion of the root of penis, for example, against its top and sides, and these inventions comprise the prior art for my invention. The invention for which a patent is here sought is of this later type (classification 600/41.) It consists of a cord and arrangements for this cord to be wound around a man's body and secured in place and so arranged that it passes across and presses in on the upper half of his penis where his penis meets his body (see FIGS. 1 and 2). The advantage of this invention over the previous art, which consist of various rigid contraptions, is that it is much simpler, much less conspicuous, is much more easily and quickly installed, is of greater help in a man's achieving and maintaining an erect penis and has a very low cost of manufacture.

### A BRIEF SUMMARY OF THE INVENTION

As an aid in remedying impotence, this invention consists of a cord and arrangements for it to be wound around a man's body and secured in place and so arranged that it passes across and presses in on the upper half of his penis where his penis meets his body thus including a very simple method of restricting the flow of blood out of his penis without interfering with blood flow into his penis nor with ejaculation.

### BRIEF DESCRIPTION OF THE SEVERAL VIEWS OF THE DRAWING

FIG. 1 shows a profile view of the invention in use in the embodiment described in claim 1.

FIG. 2 shows a front view of the invention in use in the embodiment described in claim 1.

FIG. 3 shows a front view of the invention in use in the embodiment described in claim 3.

FIG. 4 shows a profile view of the invention in use in the embodiment described in claim 4.

### DETAILED DESCRIPTION OF THE INVENTION

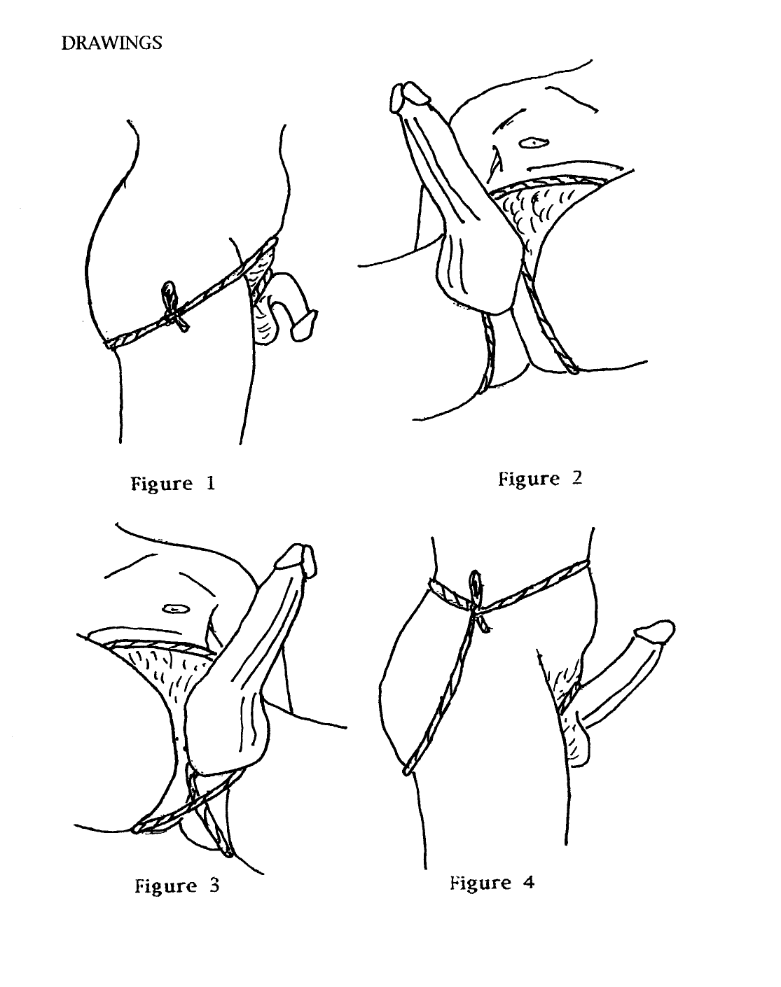

This invention consists of a cord and arrangements for it to be wound around a man's body and secured in place and so arranged that it passes across and presses in on the upper half of his penis where his penis meets his body (see FIGS. 1 and 2.) In its preferred embodiment the cord is approximately ⅛ inch in diameter and made of an elastic material, for example, it is woven with spandex thread, and when installed, firmly presses against the penis without using up the elasticity of the cord. In its various forms, it may be installed as follows: The man is in a standing position and the cord is secured around his body above the level of his penis and possibly as high as his waist. He or his partner reaches between his legs, grasps a strand of the cord lying against his back, pulls it down, then forward to in front of him, then up and over his penis and testicles and releases it so that it lies across the upper part of the root his penis. This invention provides a very simple, inconspicuous, easily and quickly installed aid, without side effects, to a man's achieving and maintaining an erect penis. In some men suffering from impotence its installation allows them to proceed with sexual activity in a normal manner. In others it can be an aid to the effectiveness of additional remedies.

1. I claim as my invention, as an aid to a man's developing and maintaining an erect penis, a cord made of an elastic material passing across the front of a man's body just above the level of his penis, around the backs of both his legs and then forward following the lines where his legs meet his buttocks and then up and over his penis passing where his penis joins his body (see FIGS. 1 and 2.), the cord being initially fitted and secured in place so that when in position it is fairly tight without fully using up the cords elasticity.

2. I claim as my invention, the same as claim 1 except the cord need not be elastic.

3. I claim as my invention, the same as 1 or 2 except the cord is crossed before being brought up and over the penis (see FIG. 3.)

4. I claim as my invention, a loop secured around the waist with an additional strand, attached at each side of the waist loop, which goes down between the legs and up and over as in claims 1, 2 or 3 (see FIG. 4).

5. I claim as my invention, as an aid to a man's developing and maintaining an erect penis, a cord when installed and secured to a man's body, passes over and presses on the upper portion of his penis where his penis meets his body.
